(19) United States
(12) Patent Application Publication
(10) Pub. No.: US 2026/0298906 A1
(43) Pub. Date: Oct. 1, 2026

(54) APPARATUS FOR THREE-DIMENSIONAL SENSING

(71) Applicant: RAMOT AT TEL-AVIV UNIVERSITY LTD., Tel Aviv (IL)

(72) Inventors: Ben Meir MAOZ, Tel Aviv (IL); Uri ASHERY, Tel Aviv (IL); Noam RAHAV, Tel Aviv (IL)

(21) Appl. No.: 18/881,854

(22) PCT Filed: Jul. 6, 2023
(86) PCT No.: PCT/IL2023/050707

§ 371 Date: Jan. 7, 2025

Related U.S. Application Data
(60) Provisional application No. 63/358,921, filed on Jul. 7, 2022.

(51) Int. Cl. G01N 33/483; C12M 1/34

(52) U.S. Cl. CPC G01N 33/4836; C12M 41/46

(57) ABSTRACT

An apparatus for three-dimensional sensing is disclosed. The apparatus comprises an electrode housing, comprising: two or more walls capable of encompassing a 3D model, wherein at least two walls are detachably connected to each other, forming at a connected state an angle greater than 0 degrees, between surfaces of each of the two or more walls; and wherein each wall comprises an electrode array, extending out from the wall towards the 3D model, configured to sense electrical activity of the 3D model.

### CROSS REFERENCE TO RELATED APPLICATIONS

This application claims the benefit of priority of U.S. Provisional Patent Application No. 63/358,921, filed Jul. 7, 2022 titled “APPARATUS FOR THREE-DIMENSIONAL SENSING”, the contents of which are all incorporated herein by reference in its entirety.

### FIELD OF THE INVENTION

The present invention relates generally to in vitro apparatuses. More specifically, the present invention relates to multi-sensory platforms for in vitro sensing of brain models.

### BACKGROUND OF THE INVENTION

Advances in the field of tissue engineering enable better emulation of the human body by creating multi-cellular three-dimensional (3D) in-vitro models. Three-dimensional (3D) bioprinting has emerged as a promising approach for engineering the in vitro models that closely recapitulate the anatomy and physiology of the human body. With the aid of computer-aided design (CAD) software, bio-printed models can be produced with high spatial resolution and control, with biocompatible materials, biochemical cues, and heterogeneous cell populations deposited in precise configurations. Moreover, bioprinting can be used to incorporate vascular networks into tissue, as well as to match organs' biomechanical properties.

To better understand brain functionality and find therapeutic solutions, the neurovascular unit (NVU) is being investigated worldwide. While complex 3D brain structures are fabricated by bio-printing and other methods, the monitoring of these models is very challenging and there is a lack of sensing solutions. Currently, sensors in the field include trans-endothelial electrical resistance (TEER) sensors and multi-electrode array (MEA) sensors. However, current solutions only measure in vitro models in two-dimensional space.

Thus, a solution is needed that provides a multi-sensory platform that is capable of being customized for different types of in vitro brain models, including 3D in vitro models.

### SUMMARY OF THE INVENTION

Embodiments of the present invention are directed to an apparatus for three-dimensional sensing, comprising: an electrode housing, comprising: two or more walls capable of encompassing a 3D model, wherein at least two walls are detachably connected to each other, forming at a connected state an angle greater than 0 degrees, between surfaces of each of the two or more walls; and wherein each wall comprises an electrode array, extending out from the wall towards the 3D model, configured to sense electrical activity of the 3D model.

In some embodiments, the 3D model is a 3D in vitro model. In some embodiments, each wall surface has area ranging from 100 mm² to 10000 mm².

In some embodiments, locations of the electrodes in the array are determined based on the 3D model. In some embodiments, the locations are determined to sense the electrical activity of at least one of: one or more elements and one or more organs in the 3D model.

In some embodiments, the at least two walls are comprised of polydimethylsiloxane (PDMS). In some embodiments, the at least two walls are configured to hinge on one or more connecting edges of each wall.

In some embodiments, the sensing apparatus further comprises an electrode plate, wherein the electrode plate is configured to electrically connect the array of electrodes in the housing with one or more external electrodes via at least one connector.

In some embodiments, the sensing apparatus comprises at least two types of electrode arrays. In some embodiments, a first electrode array comprises Multi-Electrode-Array (MEA) for assessment of neural function in the 3D model. In some embodiments, a second electrode array comprises es Trans-Endothelial-Electrical-Resistance (TEER) electrodes for monitoring vasculature selectivity in the 3D model.

In some embodiments, the sensing apparatus further comprises a controller configured to: receive, from the electrode array, a signal indicative of electrical activity in the 3D model; and measure the electrical activity in the 3D model based on the received signal.

In some embodiments, the 3D model is a model of brain tissue, and the signal is indicative of neural activity in the brain tissue.

### BRIEF DESCRIPTION OF THE DRAWINGS

The subject matter regarded as the invention is particularly pointed out and distinctly claimed in the concluding portion of the specification. The invention, however, both as to organization and method of operation, together with objects, features, and advantages thereof, may best be understood by reference to the following detailed description when read with the accompanying drawings in which:

FIGS. 1A and 1B show illustrations of a three-dimensional sensing apparatus according to some embodiments of the invention;

FIG. 1C includes several images and illustrations of a detailed example for a three-dimensional sensing apparatus according to some embodiments of the invention;

FIGS. 2A and 2B show illustrations of a three-dimensional sensing apparatus according to some embodiments of the invention;

FIGS. 3A and 3B show illustrations of a three-dimensional sensing apparatus according to some embodiments of the invention;

FIG. 3C includes two images and graphs of nonlimiting example of electrodes for the sensing apparatus according to some embodiments of the invention;

FIGS. 4A and 4B show illustrations of neural activity measurements as measured by a three-dimensional sensing apparatus according to some embodiments of the invention;

FIG. 5 shows images and graphs of a vascular modeling using the apparatus according to some embodiments of the invention; and

FIG. 6 shows illustrations and images of integration of a 3D bioprinted tissue constructs with custom configurations of an apparatus according to some embodiments of the invention.

It will be appreciated that for simplicity and clarity of illustration, elements shown in the figures have not necessarily been drawn to scale. For example, the dimensions of some of the elements may be exaggerated relative to other elements for clarity. Further, where considered appropriate, reference numerals may be repeated among the figures to indicate corresponding or analogous elements.

### DETAILED DESCRIPTION OF THE PRESENT INVENTION

One skilled in the art will realize the invention may be embodied in other specific forms without departing from the spirit or essential characteristics thereof. The foregoing embodiments are therefore to be considered in all respects illustrative rather than limiting of the invention described herein. Scope of the invention is thus indicated by the appended claims, rather than by the foregoing description, and all changes that come within the meaning and range of equivalency of the claims are therefore intended to be embraced therein.

In the following detailed description, numerous specific details are set forth in order to provide a thorough understanding of the invention. However, it will be understood by those skilled in the art that the present invention may be practiced without these specific details. In other instances, well-known methods, procedures, and components have not been described in detail so as not to obscure the present invention. Some features or elements described with respect to one embodiment may be combined with features or elements described with respect to other embodiments. For the sake of clarity, discussion of same or similar features or elements may not be repeated.

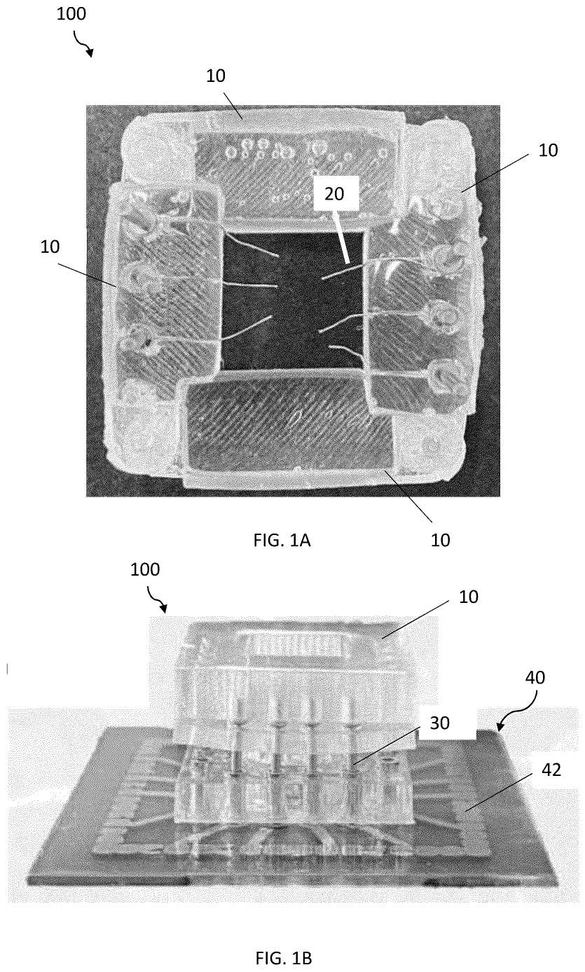

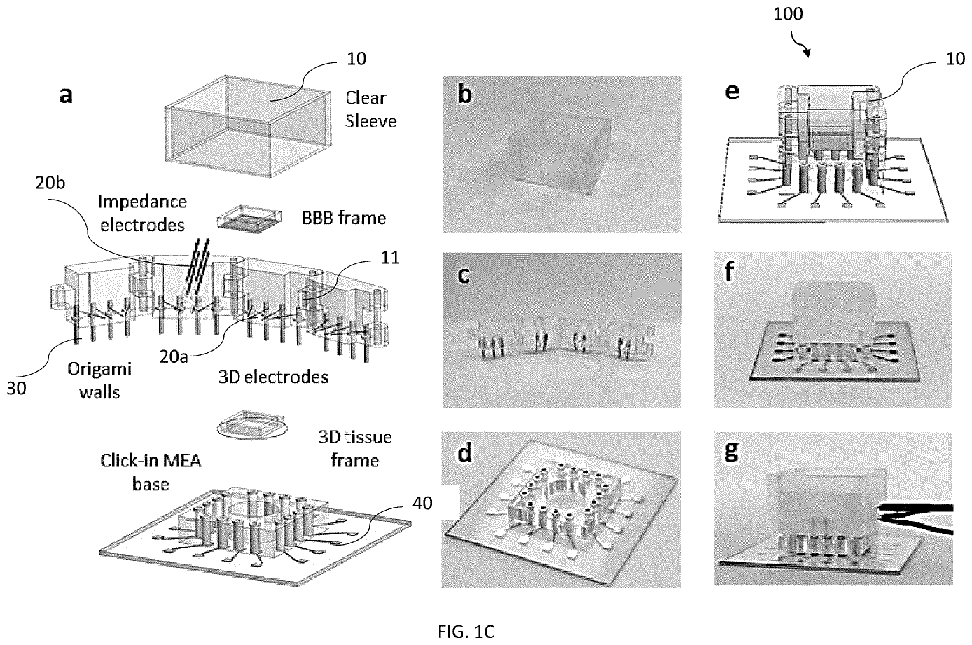

Reference is now made to FIGS. 1A and 1B showing illustrations of a three-dimensional sensing apparatus according to some embodiments of the invention.

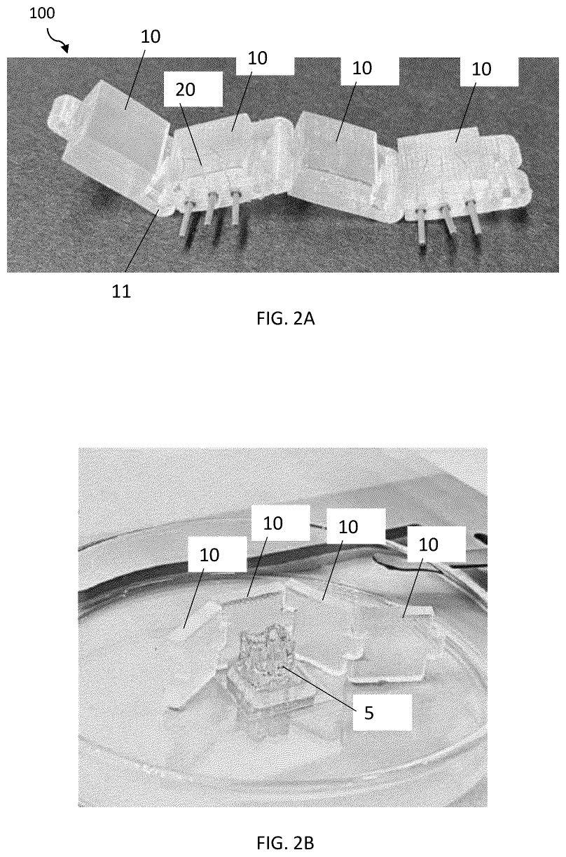

An apparatus 100 may include an electrode housing, where the electrode housing may include two or more walls 10 capable of encompassing a three-dimensional model. In some embodiments, the walls 10 may be detachably connected to each other; where walls 10 form, at a connected state, an angle greater than 0 degrees (e.g., 90 degrees) between surfaces of each of the walls 10. In some embodiments, walls 10 may be configured to hinge on one or more connecting edges of each wall 10, further illustrated and discussed herein with respect to FIGS. 2A and 2B.

In some embodiments, each wall 10 surface has dimensions (e.g., width, height) ranging from 10 mm to 20 mm, 20 mm to 50 mm, 50 mm to 100 mm, and any range and value herein between. In some embodiments, each of the surfaces of the two or more walls has an area ranging from 100 mm² to 10000 mm². In some embodiments, each wall 10 may be fabricated from an elastomeric material, for example, polydimethylsiloxane (PDMS). A structure of wall 10 may be selected based on desired embedding with an electrode array, further discussed herein.

In some embodiments, each wall 10 comprises an electrode array 20, extending outward from each respective wall 10 towards a sample (e.g., a 3D model, further illustrated and discussed with respect to FIGS. 2A and 2B herein). Electrode arrays 20 may be configured to sense electrical activity of the sample (e.g., a biological tissue, a brain tissue, a printed biological tissue, etc.), further discussed herein.

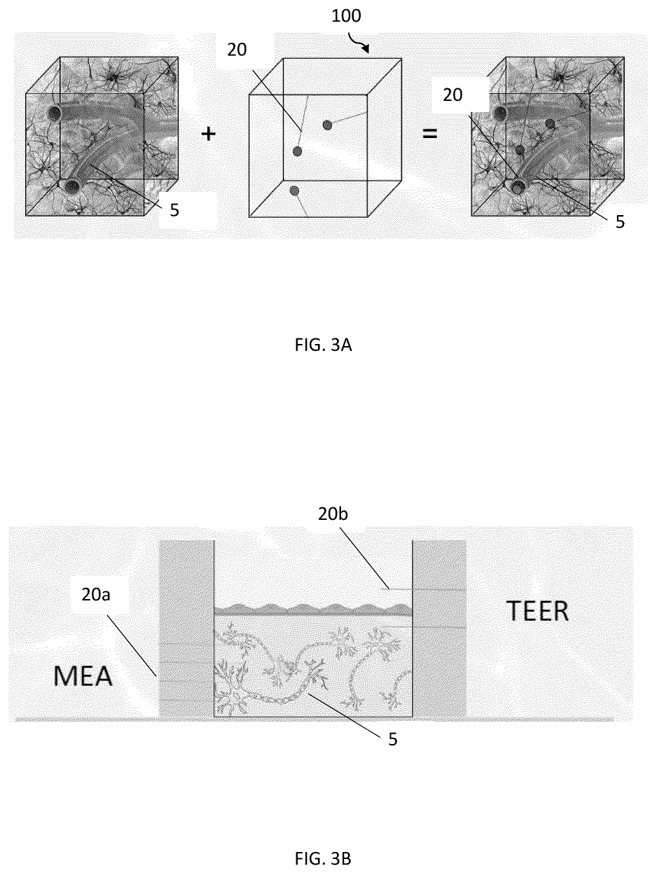

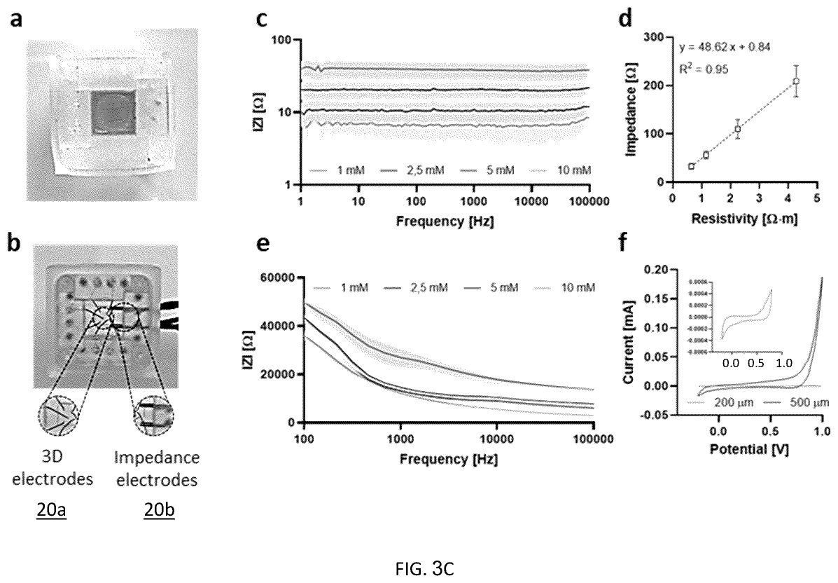

In some embodiments, electrode arrays 20 may be, or may include at least two types of electrode arrays. In some embodiments, electrode arrays 20 may include a first electrode array (e.g., array 20a illustrated in FIGS. 1C, 3B and 3C) and a second electrode array (e.g., array 20b illustrated in FIG. 3B), where the first electrode array may be selected from non-limiting example including a Multi-Electrode-Array (MEA), and the second electrode array may be selected from non-limiting example including a Trans-Endothelial-Electrical-Resistance (TEER) electrode array. In some embodiments, electrode arrays 20 may be selected from conventional stainless-steel electrodes with gold pins, as used in the art.

In some embodiments where electrode array 20/20a is selected from an MEA, electrode array 20 may be configured to assess neural function (e.g., neural electrical activity) in the sample.

In some embodiments, where electrode array 20/20b is selected from a TEER, electrode array 20 may be configured to monitor vasculature selectivity of the sample. In some embodiments, electrode array 20 may be configured to identify and characterize permeability differences in the sample.

In some embodiments, apparatus 100 may further include an electrode plate 40, as illustrated in FIG. 1B. In some embodiments, electrode plate 40 may be configured to electrically connect the electrode array (e.g., electrode arrays 20) with one or more external electrodes (e.g., external electrodes 42) via at least one connector 30. In some embodiments, external electrodes 42 may be fabricated on electrode plate 40 via gold-plating, as known in the art. The at least one connector 30 and external electrodes 42 may be composed of gold, as known in the art in order to provide high conductivity while transferring electrical signals (e.g., electrical signals as received by electrode arrays 20). In some embodiments, external electrodes 42 may be configured to send sensed electrical activity of the sample (e.g., as sensed by electrode arrays 20) to a controller, as further discussed herein.

In some embodiments, apparatus 100 may further include a controller (not illustrated), where the controller may be configured to: receive, from electrode arrays 20, a signal indicative of electrical activity in the sample (e.g., the 3D model), and measure the electrical activity in the sample based on the received signal. In some embodiments, the controller may be, or may include, one or more processors configured to receive signals from electrode arrays (e.g., electrode arrays 20). In some embodiments, the controller may be configured to send a measurement of electrical activity, as determined by the controller, to an external device, for example, in order to record electrical activity of the 3D model. In some embodiments, where the 3D model is a model of brain tissue (as further illustrated and discussed herein with respect to FIGS. 2A and 2B), the signal as received from electrode arrays 20 may be indicative of neural activity in the brain tissue.

Reference is now made to FIG. 1C which includes illustrations and images of a nonlimiting example of a three-dimensional sensing apparatus according to some embodiments of the invention.

FIG. 2C(a) shows an exploded view of apparatus 100. The apparatus includes the following parts: walls 10 for example, a set of four Polydimethylsiloxane (PDMS) walls 10 (also shown in FIG. 1C(c)), connected to each other with hinges 11 so as to fold around a 3D model. Each wall contains up to four 3D Multielectrode Array (MEA) electrodes 20a (e.g., 16 electrodes), which protrude from walls 10 in predetermined positions. Positioning may be achieved as follows: For each electrode in the array, the electrode is wrapped around a pin and then the pin is inserted through the bottom of the corresponding wall 10 in the designated position. Then, the electrode is guided to the desired position along the outside of wall 10 with the aid of an electrode positioning template, which has been custom-produced (3D-printed) according to the CAD model (shown in FIG. 3C).

In some embodiments, electrodes 20a fit into a click-in MEA plate 40 (FIG. 2d), whose structure matches that of a standard 60-electrode MEA plate. This design may enable apparatus 100 to be connected to a commercial MEA data acquisition system (e.g., MEA2100-Mini headstage, Multi Channel Systems). Plate 40 can be used by multiple apparatuses 100 as it can be plugged in/out for the electrical measurement. In some embodiments, the 3D model may be mounted (e.g., bioprinted directly) onto a 3D tissue frame (e.g., a framed glass coverslip that enables optical inspection under the microscope)—that is inserted into the center of plate 40. Device 100 walls 10 may then folded around the model and connected to plate 40 using connectors 30.

FIG. 1C(b) shows an image of a clear sleeve made of a hard frame material is then placed as a sheath around the assembled apparatus. The sleeve may apply an even pressure on apparatus 100 walls, thereby preventing any medium leakage. In tests in which the assembled apparatus 100 was filled with dyed water and encased in the sleeve (FIG. 3C(a)), apparatus 100 remained leak-tight over the course of the entire observation period (20 days).

In addition to the 3D MEA electrodes, device 100 may accommodate different types of sensors for specific applications, such as sensors for measuring, impedance, temperature or pH and the like. In the nonlimiting example shown in FIG. 1C, and in order to test a model of the brain NVU, the device includes an array of impedance electrodes 20b. Such a device may enable to co-culture neuronal cells in conjunction with a microvascular network (e.g., a Blood-Brain Barrier (BBB)) which is grown separately on a standalone BBB frame. To integrate the BBB into the assembled apparatus 100, it was placed directly on top of a 3D neuronal sample. To measure the vascular barrier function of the BBB, four wire-type impedance electrodes 20b were introduced through one of the apparatus walls in a tetrapolar configuration, with two electrodes above and two below the integrated BBB frame. (Four small openings are made in the clear sleeve, to enable the electrodes to pass through and penetrate the wall, as shown in FIG. 1C(g).)

Reference is now made to FIGS. 2A and 2B showing illustrations of a three-dimensional sensing apparatus according to some embodiments of the invention.

In some embodiments, walls 10 of apparatus 100 may be configured to hinge (e.g., swivel via hinges 11) on one or more connecting edges of each wall 10, as illustrated in FIG. 2A showing an unfolded state of apparatus 100. In some embodiments, an unfolded state may be characterized by relative angles of connected walls 10 set at greater than 90 degrees, for example. In some embodiments, an apparatus 100 may be configured in an unfolded state as illustrated, in order to encompass a 3D model by folding around the 3D model, as illustrated in FIG. 2B. Walls 10 may be hinged around the 3D model as illustrated in FIG. 2B, forming a complete enclosure (e.g., a complete square, as illustrated in FIG. 1A).

In some embodiments, a sample as discussed herein may be selected from a 3D model 5. In some embodiments, 3D model 5 may be a 3D in vitro model. In some embodiments, 3D model 5 may be a model of brain tissue. For example, 3D model 5 may be a model of co-cultured 3D neurons and glial cells with endothelial cells. In such embodiments, electrode arrays 20 may be arranged in relation to a structure of model 5, in order to gain access to the desired cellular function of model 5, as illustrated and discussed herein with respect to FIGS. 3A and 3B.

Reference is now made to FIGS. 3A and 3B showing illustrations of a three-dimensional sensing apparatus according to some embodiments of the invention. In some embodiments, as illustrated in FIG. 3A, an arrangement of electrode arrays 20 may be based on a structure of model 5, in order to measure electrical activity of specific locations of model 5. For example, electrode arrays 20 may be placed at locations of blood vessels or parenchymal tissues of model 5. As illustrated in FIG. 3B, array 20 may include a combination of MEA electrode array 20a and Transepithelial/Transendothelial electrical resistance (TEER) electrode array 20b, which may be used to sense electrical activity of model 5. In some embodiments, an apparatus 100 may combine MEA electrode array 20a and TEER electrode array 20b to simultaneously assess neural function (e.g., via MEA electrodes 20) and monitor the selectivity of brain vasculatures (e.g., via TEER electrodes 20). In some embodiments, other suitable sensor electrodes may replace the TEER and/or MEA electrodes.

Reference is now made to FIG. 3C which shows two images and graphs showing nonlimiting examples for the characterization of the electrodes for the sensing apparatus according to some embodiments of the invention.

Two types of electrodes 20a and 20b included in apparatus 100 were characterized. The apparatus included impedance TEER electrodes 20b and 3D MEA electrodes 20a both made from stainless steel, shown together the images of FIGS. 3C(a and b). The MEA electrodes have a diameter of 200 and the TEER electrodes has a diameter of 500 μm.

In the nonlimiting example shown in FIG. 3C the electrodes were characterized by electrochemical impedance spectroscopy. The electrode impedance was recorded using a commercially available potentiostat (MTZ-35, Biologic Science Instruments) driven by EC-lab software. The impedance was measured using a sinusoidal excitation signal with an amplitude of 10 mV in the frequency range of 10 Hz to 100 kHz. Impedance measurements were carried out using a tetrapolar configuration by connecting the upper left electrode as the working electrode, the lower left electrode as the counter electrode, and the upper right and the lower right electrodes as reference electrodes. To check if electrodes were responsive to changes in medium conductivity, we recorded the electrode impedance of phosphate-buffered saline solution (PBS) at different molarities: 10, 5, 2.5, and 1.25 mM. The device cell constant was calculated according to the following expression in equation (1):

$$K = \frac{R}{ρ} [ {m}^{- 1} ] \qquad ( 1 )$$

where R is the electrode access resistance in Ω and ρ the electrolyte resistivity in Ω·m.

The impedance spectra of both types of electrodes (TEER and MEA) were recorded after filling the device with saline solutions at different molarities (FIG. 3C(a)). Since it was necessary to eliminate the electrode polarization impedance, a tetra-polar electrode configuration was used, which allowed taken into consideration only the impedance of the electrolyte. Therefore, the impedance spectra resemble a flat line with decreasing impedance as electrolyte conductivity increases (as shown in FIG. 3C(c)).

In some embodiments, the device cell constant (K) was obtained from the linear and proportional to the electrolyte resistivity, which result in a value of 48.62 m−1 (FIG. 3C(d)). In contrast to tetra-polar measurements, bipolar measurements (FIG. 3C(e)) exhibited two regions: at high frequencies, the impedance was directly dependent on the ion solution conductivity and electrode resistance, whereas at low frequencies, the signal was attenuated in a nonlinear fashion by the double-layer capacitance (FIG. 3C(e)). In addition, the active areas of the stainless-steel electrodes were measured by cyclic voltammetry (FIG. 3C(f)); the 500 μm electrode was shown to have a much larger active surface area compared to the 200 μm electrode.

### Experimental Result

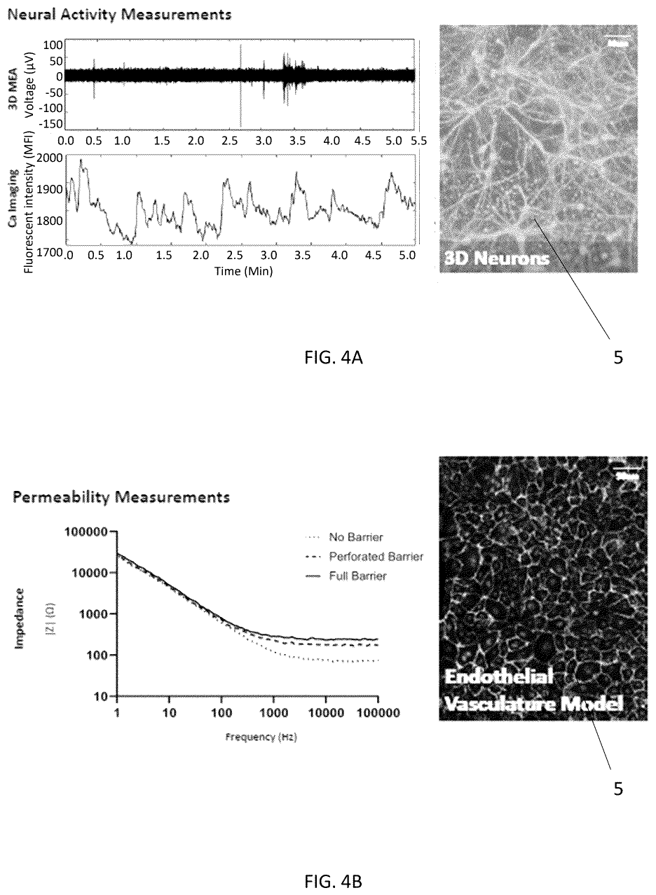

Reference is now made to FIGS. 4A and 4B that show neural activity measurements as measured by a three-dimensional sensing apparatus (e.g., apparatus 100) according to some embodiments of the invention.

An image 50 of the neural activity model (e.g., of a 3D in vitro model 5), and the corresponding 3D MEA and Ca imaging as a function of time, are shown in FIG. 4A. These measurements may be measured by MEA electrodes 20a of apparatus 100. An image 55 of the endothelial vascular model made from permeability measurements of model 5, and the impedance measurement as a function of the frequency are illustrated in FIG. 4B. These measurements may be measured by TEER electrodes 20b of apparatus 100.

### Endothelial Cell Culture

Brain microvascular endothelial cells (BMECs) were differentiated from human induced pluripotent stem cells (hiPSCs; BGU003imTOR passage 16-18), some modifications 26. Specifically, hiPSCs were seeded on Matrigel-coated plates (Corning, 354234) at 20,800 cells/cm2 24 hours prior to differentiation. We initiated differentiation by culturing the cells in DMEM/F12 medium supplemented with 20% Knockout serum (Gibco, 10828010), 1% non-essential-A amino-acids (Biological Industries, 01-340-1B), 1 mM L-glutamine (Gibco), 216 μM β mercaptoethanol (Gibco, 31350-010), 100 U/ml penicillin, and 100 μg/ml streptomycin (Biological Industries, 03-031-1B). Medium was changed daily. After 4 days, cells were seeded on top of a PC membrane (in the BBB frame; see above) coated with 400 μg/ml collagen (Sigma-Aldrich, C5533) and 100 μg/ml fibronectin (Corning, 356008) for 4 h at 37° C. Cells were cultured in serum-free medium (Gibco, 11111044) supplemented with 20 ng/ml bFGF (Peprotech, 100-18B), 10 μM retinoic acid (Sigma-Aldrich, R2625), B27 (Gibco, 12587010) and 100 U/ml penicillin, and 100 μg/ml streptomycin. After two days of seeding, medium was switched to serum-free medium supplemented with B27 and 100 U/ml penicillin, and 100 μg/ml streptomycin. Medium was changed every other day.

### Neuronal Cell Culture

### Derivation and Analysis of Cerebral Organoids.

Cerebral organoids were generated. iPSC lines were cultured in Matrigel (100 μg ml−1; FAL354234, Lapidot) coated tissue culture plates (Corning) in Nutristem (05-100-1A, Biological Industries). For cortical differentiation the cells were grown to 60-80% confluency and dissociated into a single cell suspension by first incubating with 1 ml EDTA (0.5 mM) for 2 min at 37° C., followed by substituting the EDTA with 1 ml Accutase™ (per 60-mm culture dish) and incubation for 3 min at 37° C. The cells were then triturated 10-15 times using p1000 tips until single cells were obtained. The single-cell suspension was first washed with Nutristem and then with hESC medium, containing DMEM/F12 (01-170-1A. Biological Industries), 20% (v/v) Knockout Serum Replacement (01828010; Gibco-Rhenium), 1 mM Glutamax (35050038; Gibco), 100 μM MEM nonessential amino acids (01-340-1B; Biological Industries), 1% PenStrep (03-031-1, B; Biological Industries) and 0.1 mM β-mercaptoethanol (M3148; Sigma-Aldrich) and supplemented with FGF2 (4 ng ml−1) and ROCK inhibitor (50 μM; Tocris), after which the cells were centrifuged at 270 g for 5 min.

As should be understood by the one skilled in the art apparatus 100 may be used to sense two dimensional organoids or three dimensional organoids.

Single cells were counted and the volume of the hESC medium was adjusted along with FGF2 and ROCK inhibitor to a concentration of 9,000 cells per 150 μl. Suspended single cells were plated on a 96-well U-bottom low-attachment plate (Corning). The plate was inspected for cell aggregation and formation of EBs on day 1. On day 2, half of the medium was aspirated without disturbing aggregates and 150 μl hESC medium was added to a total of 225 μl hESC medium along with the appropriate inhibitor molecules-SB-431542 (10 μM), LDN (250 ng ml−1) and XAV-939 (3.3 μM). FGF2 and ROCK inhibitor were withdrawn once the EBs reached a size of approximately 350 μm. On day 4, 150 μl medium was removed and replaced with fresh 150 μl hESC medium along with the corresponding inhibitor molecules. On day 6, the organoids were transferred into a low-attachment 24-well plate along with N2 neural induction medium (DMEM/F12, 1 mM Glutamax, 100 μM MEM nonessential amino acids, 1% PenStrep and 1% N2 supplement (17502048; Rhenium)). Every alternate day, 300 μl medium was aspirated and replaced by an equal volume of fresh N2 medium along with factors until day 11. On day 11, the organoids (500-600 μm in size) were embedded in 30 μl Matrigel droplets and incubated for 30 min in the incubator, after which they were transferred into a six-well low-attachment plate containing N2/NB medium (1:1) along with 1% B27 without RA using a sterile spatula (NB medium containing Neurobasal medium (21103-049; Rhenium), 1 mM Glutamax, 100 μM MEM nonessential amino acids, 1% PenStrep and 0.1 mM β-mercaptoethanol). On day 13, a medium change was made using the same medium from day 11. On day 15, the entire supernatant medium was removed and replaced with fresh medium containing N2/NB medium along with 1% B27 with RA; the organoid dishes were transferred onto an orbital shaker and the medium was changed daily. For long-term organoid culture the medium was changed every 2d.

### Live/Dead Assay

Calcine (C1430, Life Technologies) and ethidium-bromide (1613016, Bio-Rad) were used to mark live and dead cells, respectively. Hoechst (Hoechst-33342 H3570, Rhenium) was used to stain the nuclei of cells. The cells were incubated for 30 minutes at 37° C. Imaging was carried out using an inverted confocal microscope (Olympus FV3000-IX83).

### Ca⁺² Imaging

Imaging was carried out using an inverted confocal microscope (Olympus FV3000-IX83).

### Immunohistochemistry

For immunohistochemistry (IHC) of endothelial cells, BMECs were stained with fluorophore-labeled antibodies directed against ZO-1 (CST-13663S, Cell Signaling) or VE cadherin (CST-2500S, Cell Signaling). For 3D neuron cultures, cells were stained with fluorophore-labeled antibodies directed against beta-tubulin (ab18207, Abcam), and GFAP (G3893-100UL, Sigma-Aldrich). After staining, both types of cells were transferred to a 24-well plate and washed three times with PBS. Subsequently, cells were fixed with 4% paraformaldehyde in PBS at room temperature for 10 min for 2D endothelial cell culture and 30 min for 3D neuronal cell cultures. After fixation, cells were washed again three times with PBS. Next, cells were permeabilized with 1% Triton X-100 in PBS for 20 min. Then, after three washing steps with PBS, cells were blocked with 1% bovine serum albumin in PBS. The cells were incubated with primary and secondary antibodies in blocking buffer for 1 h and 2 h, respectively, with three washing steps in between. After another three washing steps with PBS, nuclei were stained with DAPI (DAPI Fluoromount-G® 0100-20, Enco Scientific Services). All steps were performed at room temperature. Imaging was carried out using an inverted confocal microscope (FV3000-IX83, Olympus) and fluorescent microscope (BX60MF5, Olympus).

### Measurements

### Impedance Measurements

The impedance of endothelial cells grown on the PC membrane in the BBB frame was recorded using a commercially available potentiostat (MTZ-35, Biologic Science Instruments) driven by EC-lab software. The impedance was measured using a sinusoidal excitation signal with an amplitude of 10 mV in the frequency range of 10 Hz to 100 kHz. Impedance measurements were carried out using a tetrapolar configuration as described in the subsection titled “Impedance sensor characterization”. Impedance measurements were performed outside the incubator for a maximum of 1.5 min so as to avoid compromising cell culture.

### Impedance Data Analysis

The impedance measurements, obtained from the endothelial cells as described above, were fitted to an equivalent electric circuit using the least-squares method in Python. The equivalent circuit is composed of a constant phase element (CPE) in parallel with a resistor (RTEER) and in series with another resistor (Rs). Three well-differentiated regions can be distinguished across the frequency spectrum. At high frequencies (>105 Hz), the impedance is controlled by the cell culture medium conductivity (Rs). At intermediate frequencies (100-105 Hz), impedance is associated with the cell layer capacitance, represented by a CPE. At low frequencies (<100 Hz), impedance is determined by the sum of two ion conductive pathways—the paracellular and transcellular resistance—which is in turn associated with the transepithelial/endothelial electrical resistance (TEER). The mathematical expression for the CPE is given by equation 2

$${Z}_{CPE} = \frac{1}{{k ( j ω )}^{α}} \qquad ( 2 )$$

where j is the imaginary unit, ω is the angular frequency, k is the system admittance, and α is an exponent corresponding to 0 or 1 for an ideal resistor or capacitor, respectively. The cellular layer capacitance (Ccl) was estimated using the following equation 3:

$${C}_{cl} = \frac{{( K \cdot {R}_{TEER} )}^{\frac{1}{α}}}{{R}_{TEER}} \qquad ( 3 )$$

### MEA Measurements

The MEA2100-system (MEAs, Multi Channel Systems, Reutlingen, Germany), driven by Multi-Channel Experimenter Software, was used to record electrogenic activity of neuron cells. Our custom-made click-in MEA base (FIGS. 2a and d) was placed on the MEA2100 headstage, which links the MEA recording system and the 3D sensing device.

### Bioprinting

A SolidWorks was used to design 3D structures for bioprinting. The printing properties were set using an open-source slicer program (Slic3r) from which a g-code file was generated. A BIO-X printer (S-10001-001, Cellink) was used for printing the gel constructs using CELLINK-Start gel (s20030002, Cellink). To produce the printed models, the two inks were sequentially printed within the PDMS 3D tissue frame, supported by a custom-made platform. Food coloring was used to color the ink to enhance the visibility of the different structures embedded within the printed models. Inks were loaded and centrifuged to remove bubbles into 3-ml syringes with a nozzle diameter of 0.410 mm. Next, inks were deposited by applying a pressure ranging from 30 to 38 mPa and with a printing speed ranging from 1 to 2 mm/s at room temperature. After each model was printed, the origami walls of a corresponding apparatus 100 (with custom-designed electrode configuration) were folded around the model, thereby inserting electrodes in the desired positions.

### Results

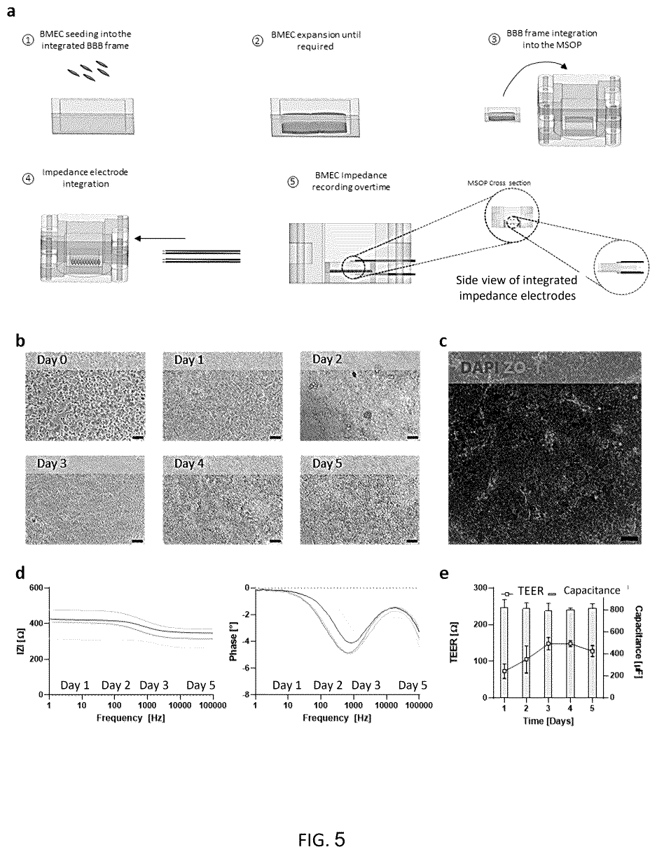

Reference is now made to FIG. 5 which includes various measurements and results from a vascular modeling done using the apparatus according to some embodiments of the invention. FIG. 5 a) is an illustration of the vascular component cell culture and barrier function monitoring setup. BMECs are grown on top of the BBB frame to form a confluent monolayer. Then, the BBB frame is introduced inside the apparatus. For barrier function assessment, two electrodes are positioned on either side of the membrane to perform impedance measurements in a tetrapolar configuration. FIG. 5 b) is a microscopy images showing the development of hiPSC-derived BMECs from day 1 to day 5 of culture (scale bar is 100 μm). FIG. 5 c) is a maximum intensity projection of a confocal image of an immunofluorescent labeled vascular monolayer for DAPI (in blue) and ZO-1 (in green) at day 5 of cell culture (scale bar is 100 μm). d) Experimental impedance data recorded during 5 days of hiPSC-derived BMEC culture inside the apparatus. The vascular barrier function was measured over the frequency range of 1 to 105 Hz. The impedance at low frequencies (<10² Hz) corresponds to the TEER and the overall device resistance, and the negative slope at intermediate frequencies (10²-10⁴ Hz) is related to the cellular capacitance. e) TEER and capacitance values of hiPSC-derived BMECs over time, obtained by fitting the impedance spectra to an equivalent electric circuit.

The apparatus was designed to enable a microvasculature network, in this case, a BBB, to be prepared as a standalone unit and then integrated into the 3D model when required. The barrier function of the BBB can then be assessed using impedance electrodes 20b, arranged in a tetrapolar configuration, with two electrodes above and two below the barrier.

To demonstrate this functionality, the BBB was prepared by culturing BMECs in the designated BBB frame. The cells were seeded at high density in order to enable a confluent vascular monolayer to form rapidly, as shown in FIG. 5(b). Indeed, it shown that the BMECs started to form a tight monolayer with very defined cell-to-cell borders at early stages of the cell culture (due to the increase in TEER). Moreover, immunofluorescence imaging confirmed the presence of ZO-1 in BMECs forming a tight confluent monolayer as shown in FIG. 5(c).

In the next step impedance measurements were used (cellular impedance) to evaluate the functionality of the BBB that was integrated with Apparatus 100. The recorded spectra of BMECs display three distinct regions, as shown in FIG. 5(d), which are indicators of different physical characteristics of biological barriers. At low frequencies (<100 Hz), the impedance is associated with transepithelial/transendothelial electrical resistance (TEER) which is an indicator of cellular barrier strength. At intermediate frequencies (10²-10⁴ Hz), the impedance is related to cellular capacitance. At high frequencies (>10⁴), the contribution is due to the medium and the membrane resistance. As shown in in FIG. 5(e), there is a constant increase in the TEER values, which start at around 75Ω after 1 DIV, and reach a peak at day 3 with an average value of 140Ω. The values remain similar for the next two days. In contrast to TEER, cellular capacitance remained stable and constant at 800 μF for all 5 days. This observation is consistent with the fact that endothelial cells are morphologically flat and do not produce 3D structures on their apical side; Therefore, once the monolayer has been formed on day 1 of culture, the cellular capacitance remains constant for the rest of the culture period.

### Combined Demonstration: Sensing in a 3D Model of the Neurovascular Unit (NVU)

Having demonstrated that apparatus 100 can be used to separately measure (i) electrical activity in a 3D neuronal model (using the 3D MEA electrodes embedded in the device walls), and (ii) barrier function in the BBB (using impedance electrodes, i.e., TEER and capacitance), the next step was to validate the two functions simultaneously in a comprehensive model of the NVU. The model was made up of a 3D culture of neuronal cells, in combination with BMECs cultured on the BBB frame, as shown in FIG. 5(a) The NVU model was embedded in apparatus 100, and both functionalities (MEA and TEER) were measured in situ. The data that were obtained correlated with the measurements acquired for the independent compartments. An increase in the barrier function was observed. Additionally, a spontaneous electrical activity of the 3D neuronal culture was also observed.

### Customized 3D Sensing for Bioprinted Tissues

A key feature of apparatus 100 is the capacity to tailor the positioning of 3D MEA electrodes to a specific 3D model. As discussed in previous sections, the desired positioning of the electrodes is achieved with the aid of an electrode positioning template, which is custom-produced using CAD software. When the walls of apparatus 100 are “folded” around the 3D model, the electrodes penetrate the tissue in the desired locations while causing minimal damage to the tissue's structural integrity.

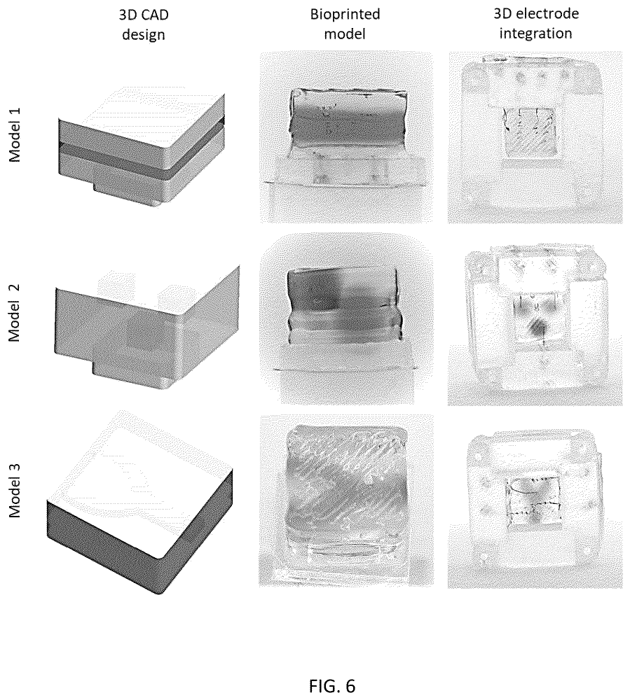

Reference is now made to FIG. 6 which shows integration of 3D bioprinted tissue constructs with custom configurations into the apparatus according to some embodiments of the invention. FIG. 6 shows three examples of bioprinted models together with customized configurations of apparatus 100 (see SI FIG. 3 for images of the electrode templates). The models were produced using hydrogel inks in different colors, representing specific regions of interest with different geometries and architectures. For example, model 1 contains a 500-μm-thick region, colored blue, at the center of the 3D structure; this region could represent a tissue barrier, for example. In this case, the electrodes are all positioned on a single wall of apparatus 100, with two electrodes above the blue region and two below it; this setup could enable measurement of the tissue barrier's permeability. Model 2 consists of three cubes, one red and two blue. In this case, there are two electrodes on one wall of the MS apparatus 100 OP, inserted in the two blue cubes, respectively, and a third electrode, on the opposite wall, is inserted in the red cube. In model 3, the blue-dyed region represents the vasculature, with a large vessel splitting into two smaller ones. This model contains four electrodes (two pairs on two opposing walls), all positioned along the vasculature.

While certain features of the invention have been illustrated and described herein, many modifications, substitutions, changes, and equivalents may occur to those skilled in the art. It is, therefore, to be understood that the appended claims are intended to cover all such modifications and changes as fall within the true spirit of the invention.

Various embodiments have been presented. Each of these embodiments may of course include features from other embodiments presented, and embodiments not specifically described may include various features described herein.

1. An apparatus for three-dimensional sensing, comprising:
an electrode housing, comprising:
two or more walls capable of encompassing a 3D model, wherein at least two walls are hingedly connected to each other, forming at a connected state an angle greater than 0 degrees, between surfaces of each of the two or more walls; and
wherein each wall of the two or more walls, comprises an electrode array, extending out from the wall towards the 3D model, configured to sense electrical activity of the 3D model.

2. The apparatus of claim 1, wherein the 3D model is a 3D in vitro model.

3. The apparatus of claim 1, wherein each of the surfaces of the two or more walls has an area ranging from 100 mm² to 10000 mm².

4. The apparatus of claim 1, wherein locations of the electrodes in the array are determined based on the 3D model.

5. The apparatus of claim 4, wherein the locations are determined to sense the electrical activity of at least one of: one or more elements and one or more organs in the 3D model.

6. The apparatus of claim 1, wherein the two or more walls are comprised of polydimethylsiloxane (PDMS).

7. The apparatus of claim 1, wherein the two or more walls are detachably connected, by one or more connecting edges of each other.

8. The apparatus of claim 1, further comprising an electrode plate, wherein the electrode plate is configured to electrically connect the electrode array in the housing with one or more external electrodes via at least one connector.

9. The apparatus of claim 1, comprising at least two types of electrode arrays.

10. The apparatus of claim 9, wherein a first electrode array comprises Multi-Electrode-Array (MEA) for assessment of neural function in the 3D model.

11. The apparatus of claim 9, wherein a second electrode array comprises Trans-Endothelial-Electrical-Resistance (TEER) electrodes for monitoring vasculature selectivity in the 3D model.

12. The apparatus of claim 1, further comprising a controller configured to:
receive, from the electrode array, a signal indicative of electrical activity in the 3D model; and
measure the electrical activity in the 3D model based on the received signal.

13. The apparatus of claim 12, wherein the 3D model is a model of brain tissue and the signal is indicative of neural activity in the brain tissue.
